(19) United States
(12) Patent Application Publication
(10) Pub. No.: US 2026/0355104 A1
(43) Pub. Date: Nov. 12, 2026

(54) COMPOSITION FOR IMPROVING SKIN CONDITION

(71) Applicant: LG HOUSEHOLD & HEALTH CARE LTD., Seoul (KR)

(72) Inventors: Jae-Young SHIN, Seoul (KR); Yu-Jin HONG, Seoul (KR); Ji-Ye PARK, Seoul (KR); Yun-Hee CHANG, Seoul (KR); Ji-Won PARK, Seoul (KR); Hye-Jin LEE, Seoul (KR); Hye-Yeon CHUN, Seoul (KR); Young-Bong KIM, Seoul (KR)

(73) Assignee: LG HOUSEHOLD & HEALTH CARE LTD., Seoul (KR)

(21) Appl. No.: 19/515,113

(22) PCT Filed: Aug. 30, 2024
(86) PCT No.: PCT/KR2024/013055

§ 371 Date: Feb. 27, 2026

(30) Foreign Application Priority Data
Aug. 31, 2023 (KR) 10-2023-0115849

(51) Int. Cl. A61K 8/9789; A61K 8/34; A61K 8/365; A61K 8/49; A61K 8/60; A61K 8/63; A61Q 19/00; A61Q 19/02; A61Q 19/08

(52) U.S. Cl. CPC A61K 8/9789; A61K 8/34; A61K 8/365; A61K 8/498; A61K 8/60; A61K 8/63; A61Q 19/00; A61Q 19/02; A61Q 19/08

(57) ABSTRACT

The present invention provides a cosmetic composition for improving skin conditions, the composition comprising bisabolol, oryzanol, extract of Reynoutria japonica, chrysin, quercetin, sodium mannose phosphate, kaempferol and pyruvate.

The composition for improving skin conditions according to the present invention exhibits excellent synergistic effects on skin barrier enhancement, pigmentation suppression, wrinkle reduction, or elasticity enhancement through the combination of specific components, and thus can be effectively utilized as a functional composition in cosmetics or topical skin preparations targeting skin tissues.

### TECHNICAL FIELD

This application claims priority to Korean Patent Application No. 10-2023-0115849, filed Aug. 31, 2023, the entire disclosure of which is incorporated herein by reference.

The present disclosure relates to a composition for improving skin conditions.

### BACKGROUND ART

The skin serves as a barrier, protecting the body from the external environment and preventing the loss of internal moisture and beneficial substances. Furthermore, it is a vital organ responsible for various physiological functions, including temperature regulation and excretion. However, various factors can cause skin cell activity to decline and skin condition to deteriorate.

Collagen and elastin, found in the dermal layer of the skin, play a significant role in skin composition, performing functions such as maintaining skin's mechanical strength, connective tissue resistance, maintaining tissue cohesion, and supporting cell adhesion. Collagen is reduced by stress, aging, and photoaging caused by UV exposure. Furthermore, elastin is known to distort its three-dimensional structure after UV exposure due to increased activity of elastase. Consequently, the decrease in collagen or decreased activity of elastin causes skin tissue to become loose and lose elasticity.

As the cell function of the epidermis declines, metabolism becomes impeded, and dead skin cells do not shed properly, resulting in excessive accumulation of dead skin cells. In this state, exposure to ultraviolet rays reduces skin elasticity and causes wrinkles. Or, if a sebum barrier fails to form on the epidermis, moisture and sebum secretion decreases, drying out the skin and leading to the formation of wrinkles.

When the skin is exposed to UV rays, it synthesizes melanin to protect itself. This synthesized melanin is transferred to keratinocytes via melanosomes. These keratinocytes undergo a 28-day turnover cycle. Normally, the melanin produced is shed by these keratinocytes every 28 days. However, if the skin cell cycle is disrupted by factors such as stress or skin aging, keratinocytes do not shed in a timely manner, resulting in pigmentation such as melasma, freckles, and age spots.

Research has continued to identify substances that can suppress skin aging caused by the various factors mentioned above and improve factors such as strengthening the skin barrier, suppressing pigmentation, and improving wrinkles and elasticity by stimulating collagen production. However, substances that demonstrate satisfactory effects in these functions are still under development, and thus, the development of new ingredients capable of improving these factors is urgently needed.

### DISCLOSURE

### Technical Problem

The problem to be addressed by the present invention is to provide a composition for improving skin conditions that exhibits improved effects in factors such as strengthening the skin barrier, suppressing pigmentation, improving wrinkles, or enhancing elasticity.

### Technical Solution

In order to address the above problem, the inventors of the present invention have made extensive research efforts and have discovered that a composition comprising bisabolol, oryzanol, extract of Reynoutria japonica, chrysin, quercetin, sodium mannose phosphate, kaempferol and pyruvate exhibits excellent skin improvement effects such as skin barrier strengthening, pigmentation suppression, wrinkle improvement, and elasticity enhancement. In particular, they have surprisingly discovered that when these ingredients are combined, the skin barrier strengthening, pigmentation suppression, and wrinkle improvement or elasticity enhancement effects are significantly increased compared to when these ingredients are used individually, thereby completing the present invention.

Accordingly, the present invention provides a cosmetic composition for improving skin conditions, comprising bisabolol, oryzanol, extract of Reynoutria japonica, chrysin, quercetin, sodium mannose phosphate, kaempferol and pyruvate.

Bisabolol used in the present invention has the IUPAC name (2S)-6-methyl-2-[(1S)-4-methylcyclohex-3-en-1-yl]hept-5-en-2-ol], also referred to as α-(−)-bisabolol or levomenol, and is a natural monocyclic sesquiterpene alcohol. Bisabolol used in the present invention may be purchased or synthesized. The bisabolol may be included in an amount of 0.00001 to 50 wt %, preferably 0.0001 to 5 wt %, based on the total weight of the composition.

Oryzanol used in the present invention is also referred to as gamma oryzanol (γ-oryzanol) and is a mixture of lipids extracted from rice (Oryza sativa). γ-Oryzanol is mainly generated from the fat fraction of rice bran and rice bran oil, and is known as a mixture of ferulic acid esters of phytosterols and triterpenoids, particularly cycloartenyl ferulate, 24-methylenecycloartanyl ferulate, and campesteryl ferrate, which are known to account for 80% of γ-oryzanol. The oryzanol used in the present invention can be obtained by extraction from rice or by commercial purchase. The oryzanol may be included in an amount of 0.00001 to 5 wt %, preferably 0.0001 to 1 wt %, based on the total weight of the composition.

The extract of Reynoutria japonica used in the present invention refers to a substance extracted from Reynoutria japonica. Reynoutria japonica (synonyms Fallopia japonica and Polygonum cuspidatum) is a perennial herbaceous plant belonging to the family Purslane in the order Polygonum, and grows in mountains and fields throughout Korea. The stem is hollow, and reddish purple spots are scattered on the stem when young. The Reynoutria japonica used in the present invention may be purchased or directly collected. The extract of Reynoutria japonica may be included in an amount of 0.00001 to 5 wt %, preferably 0.0001 to 1 wt %, based on the total weight of the composition.

The extract of Reynoutria japonica used in the present invention includes an extract obtained by extracting Reynoutria japonica, a diluted or concentrated extract, a dried product obtained by drying the extract, a crude or purified product of the extract, or a mixture or fraction thereof, and all other extracts in the form of formulations that can be formed using the extract. The extract of Reynoutria japonica used in the present invention can also be obtained by commercial purchase.

The type of extraction solvent used to extract the above-mentioned Reynoutria japonica is not particularly limited, and any solvent known in the art can be used. Non-limiting examples of the extraction solvent include water; lower alcohols with C₁ to C₄, such as methanol, ethanol, propyl alcohol, or butyl alcohol; polyhydric alcohols, such as glycerin, butylene glycol, or propylene glycol; and hydrocarbon solvents, such as methyl acetate, ethyl acetate, acetone, benzene, hexane, diethyl ether, or dichloromethane; or mixtures thereof. Preferably, water, lower alcohols, 1,3-butylene glycol, and ethyl acetate may be used alone or in combination of two or more.

In one embodiment of the present invention, the extract of Reynoutria japonica can be used after filtering to remove floating solid particles, for example, by filtering the particles using nylon or the like, or by filtering using a cryofiltration method, or the like, or after filtering, drying using freeze-drying, hot air drying, spray drying, or the like.

Chrysin used in the present invention is also referred to as 5,7-dihydroxyflavone and is a flavone extracted from plants. Chrysin used in the present invention may be purchased or synthesized. The chrysin may be included in an amount of 0.00001 to 5 wt %, preferably 0.0001 to 1 wt %, based on the total weight of the composition.

Quercetin used in the present invention is also referred to as 3,3′,4′,5,7-pentahydroxyflavone and is a plant flavonol belonging to the flavonoid group of polyphenols. Quercetin used in the present invention may be purchased or synthesized. The quercetin may be included in an amount of 0.00001 to 5 wt %, preferably 0.0001 to 1 wt %, based on the total weight of the composition.

Sodium mannose phosphate used in the present invention has the chemical formula C₆H₁₂O₉PNa and may be purchased or synthesized. The sodium mannose phosphate may be included in an amount of 0.00001 to 1 wt %, preferably 0.0001 to 0.5 wt %, based on the total weight of the composition.

Kaempferol used in the present invention is also referred to as 3,4′,5,7-tetrahydroxyflavone and is a type of natural flavonoid. Kaempferol used in the present invention may be purchased or synthesized. The kaempferol may be included in an amount of 0.00001 to 1 wt %, preferably 0.0001 to 0.5 wt %, based on the total weight of the composition.

The pyruvate used in the present invention refers to a form of pyruvic acid (C₃H₄O₃) that has a negative charge (C₃H₃O₃⁻). The cation that forms a salt with the negatively charged pyruvate may be one cation selected from the group consisting of sodium ion, potassium ion, calcium ion, magnesium ion, barium ion, aluminum ion, and mixtures thereof, but is not limited thereto. The pyruvate used in the present invention may be purchased or synthesized. The pyruvate may be included in an amount of 0.00001 to 1 wt %, preferably 0.0001 to 0.5 wt %, based on the total weight of the composition.

In one embodiment, the mixture of the above active ingredients may be included in an amount of 0.0001 to 50 wt %, preferably 0.001 to 10 wt %, based on the total weight of the composition. The effect may be better when the content of the mixture of the above active ingredients is used within the above range.

In one embodiment, when mixing the above effective ingredients, the weight ratio of bisabolol:oryzanol:extract of Reynoutria japonica:chrysin:quercetin:sodium mannose phosphate:kaempferol:pyruvate may be 1:0.05~0.5:0.01~0.1:0.005~0.05:0.005~0.05:0.001~0.01:0.0005~0.005:0.00005~0.0005, and preferably 1:0.05~0.3:0.04~0.08:0.01~0.04:0.01~0.04:0.004~0.008:0.0005~0.003:0.0001~0.0004.

In one embodiment, the cosmetic composition may include, as a solvent, one selected from the group consisting of water, lower alcohols with C₁ to C₄, polyhydric alcohols and mixtures thereof.

In one embodiment, the improving skin conditions may be one selected from the group consisting of skin barrier strengthening, pigmentation suppression, wrinkle improvement, elasticity enhancement, and combinations thereof.

The term “skin barrier” as used herein refers to a physical, chemical, or physiological barrier formed by the skin to prevent the intrusion of external substances such as pathogens and the outflow of substances to the outside, while simultaneously preventing chemical or physical damage and loss of moisture or minerals. In addition, the term “strengthening/improving the skin barrier” may refer to chemically or biologically restoring a damaged skin barrier, thereby improving the skin's function as a barrier so that the skin can effectively prevent the intrusion of external substances or the outflow of substances to the outside. This is distinct from skin moisturizing, which simply directly replenishes moisture in the skin or physically prevents moisture loss.

In relation to strengthening the skin barrier, tight junction protein ZO-1, also known as Zonula occludens-1 (ZO-1), is a 220-kD peripheral membrane protein encoded by the human TJP1 gene and belongs to the tight junction-related protein family of zonula occludens proteins (ZO-1, ZO-2, ZO-3). ZO-1 is a junction that strengthens the bond between epidermal cells. In skin with a weakened skin barrier, such as atopy or psoriasis, a phenomenon in which ZO-1 expression is reduced is found.

Regarding pigmentation, the regulation of skin pigmentation is driven by a complex melanogenic paracrine network between mesenchymal and epithelial cells, which regulates the survival and proliferation of melanocyte, and melanin production. Among melanogenic growth factors, KITLG and its receptor c-Kit play a crucial role in regulating physiological and pathological skin pigmentation by triggering the Ras/MAPK signaling pathway. Specifically, KITLG, a factor secreted from keratinocytes, acts as a c-Kit ligand that activates c-Kit, a melanin synthesis pathway. Therefore, inhibitors of KITLG have anti-pigmentation effects.

The term “skin elasticity enhancement” as used herein refers to increasing skin elasticity, inhibiting collagen-degrading enzymes and promoting collagen production to suppress or inhibit loss of skin elasticity, or alleviating already reduced elasticity. The “skin elasticity enhancement” can be achieved by strengthening the skeleton of skin cells through increased production of fibronectin and collagen. In addition, since MMP9 (matrix metalloproteinase-9) is an ECM (extracellular matrix)-degrading enzyme secreted by the epidermis and has the effect of degrading collagen in the dermis, collagen production can be promoted by inhibiting MMP9.

In one embodiment of the present invention, the cosmetic composition may include adjuvants commonly used in the cosmetic-related technical field, such as fatty substances, organic solvents, solubilizers, thickeners, gelling agents, softeners, antioxidants, suspending agents, stabilizers, foaming agents, fragrances, surfactants, water, ionic or nonionic emulsifiers, fillers, sequestering agents, chelating agents, preservatives, vitamins, blocking agents, humectants, essential oils, dyes, pigments, hydrophilic or lipophilic active agents, or lipid vesicles, and may also include any other ingredients commonly used in cosmetics.

In one embodiment of the present invention, the formulation of the cosmetic composition may be prepared as, for example, a solution, emulsion, suspension, paste, cream, lotion, gel, powder, spray, surfactant-containing cleansing, oil, soap, liquid detergent, bath agent, foundation, makeup base, essence, toner, foam, pack, softener, sunscreen cream or sun oil, etc. Preferably, the formulation of the cosmetic composition may be prepared as, for example, a skin ointment, softener toner, nourishing toner, nourishing cream, massage cream, essence, pack, emulsion or oil gel.

The present invention also provides a skin topical formulation comprising bisabolol, oryzanol, extract of Reynoutria japonica, chrysin, quercetin, sodium mannose phosphate, kaempferol and pyruvate.

In one embodiment, the skin topical formulation may refer to a solid, semi-solid, or liquid external preparation that can be easily applied to the skin by mixing an active ingredient with various bases such as oil, petrolatum, lanolin, or glycerol. The skin topical formulation is not particularly limited, but may be in the form of a powder, gel, ointment, cream, liquid, or aerosol.

In one embodiment of the present invention, the skin topical formulation may be applied to the face or other body parts through a transdermal administration method such as direct application to the skin or spraying. The terms “administration” or “application” refer to introducing the composition of the present disclosure into the skin by any suitable method. The composition may be administered through any common route as long as it can reach the target tissue, and may be administered transdermally, preferably topically. The number of times the composition is administered or applied may be appropriately determined according to the user's needs. The amount of the composition may be appropriately adjusted depending on individual differences such as age or skin condition, or on the formulation. Typically, an appropriate amount is applied to the skin once or several times a day for one week to several months.

The present invention also provides a composition comprising bisabolol, oryzanol, extract of Reynoutria japonica, chrysin, quercetin, sodium mannose phosphate, kaempferol and pyruvate for use in improving skin conditions.

The present invention also provides a method for preparing a cosmetic composition for improving skin conditions, comprising a step of mixing bisabolol, oryzanol, extract of Reynoutria japonica, chrysin, quercetin, sodium mannose phosphate, kaempferol and pyruvate.

The present invention also provides a method for improving skin conditions, comprising the step of applying to the skin a composition comprising bisabolol, oryzanol, extract of Reynoutria japonica, chrysin, quercetin, sodium mannose phosphate, kaempferol and pyruvate.

In one embodiment of the present invention, the method for improving skin conditions may include applying the composition to the skin so that the dosage of the active ingredient in the composition is 1 to 1000 mg/L, preferably 10 to 500 mg/L. In addition, the method may include applying the composition to the skin for more than a week, for example, for 1 day to 6 months, preferably for 1 week to 4 months, but the maximum application period is not limited. In addition, the method may include applying the composition to the skin 1 to 6 times a day, preferably 1 to 3 times, for example, twice a day, in the morning and in the evening.

All ingredients described in the present disclosure preferably do not exceed the maximum usage levels specified in relevant laws and regulations of Korea, China, the United States, Europe, Japan, etc. (e.g., Regulations on Cosmetic Safety Standards (Korea) or Cosmetic Safety Technical Standards (China), etc.). That is, preferably, the cosmetic composition or the skin topical formulation composition according to the present disclosure includes the ingredients according to the present disclosure within the content limits permitted by relevant laws and regulations of each country.

### Advantageous Effects

The composition for improving skin conditions of the present invention exhibits excellent synergistic effects in strengthening the skin barrier, suppressing pigmentation, improving wrinkles, and enhancing elasticity through the combination of specific ingredients. Accordingly, the composition for improving skin conditions of the present invention can be effectively used as a functional composition in cosmetics or topical skin preparations targeting skin tissue.

### DESCRIPTION OF DRAWINGS

FIG. 1 shows the results of measuring the ZO-1 expression level of each of bisabolol, oryzanol, extract of Reynoutria japonica, chrysin, quercetin, sodium mannose phosphate, kaempferol and pyruvate, and a mixture comprising these substances.

FIG. 2 shows the results of measuring the KITLG inhibitory effect of each of bisabolol, oryzanol, extract of Reynoutria japonica, chrysin, quercetin, sodium mannose phosphate, kaempferol and pyruvate, and a mixture comprising these substances.

FIG. 3 shows the results of measuring the MMP9 inhibitory effect of each of bisabolol, oryzanol, extract of Reynoutria japonica, chrysin, quercetin, sodium mannose phosphate, kaempferol and pyruvate, and a mixture comprising these substances.

### BEST MODE

Hereinafter, the present invention will be described in detail, using examples and the like, to aid understanding. However, the examples according to the present invention may be modified in various different forms, and the scope of the present invention should not be construed as being limited to the following examples. The examples of the present invention are provided to more fully explain the present invention to those of average skill in the art.

### Example 1. Preparation of a Composition for Improving Skin Conditions

A skin tonic composition was prepared by a conventional method using bisabolol, oryzanol, extract of Reynoutria japonica, chrysin, quercetin, sodium mannose phosphate (sodium mannose phosphate), kaempferol and pyruvate according to the recipe described in Table 1 below.

TABLE 1

| | | |
|---|---|---|
| Solvent | Glycerin | 50.05 |
| Solvent | Water | 26.9692 |
| Ingredient | Cetyl ethylhexanoate | 10 |
| | Dipropylene glycol | 10 |
| | Hydrogenated lecithin | 2 |
| | Bisabolol | 0.5 |
| | Butylene glycol | 0.376 |
| | Oryzanol | 0.05 |
| | Extract of Reynoutria japonica | 0.03 |
| | Chrysin | 0.01 |
| | Quercetin | 0.01 |
| | Sodium mannose phosphate | 0.003 |
| | Mannose | 0.0012 |
| | Kaempferol | 0.0005 |
| | Sodium pyruvate | 0.0001 |

### Test Example 1. Strengthening the Skin Barrier

Immunocytochemistry was performed to measure the expression level of ZO-1, a major protein constituting the barrier. HaCaT cells were seeded in 24-well plates and incubated at 37 °C, 5% CO₂ for 24 hours. The sample was dissolved in 1 mL of medium and treated to the cells, and then incubated for another 24 hours at 37 °C, 5% CO₂. After washing with PBS, the cells were fixed with 4% paraformaldehyde for 10 minutes at room temperature. The cells were then permeabilized with PBS comprising 0.1% Triton X-100 and blocked with PBS comprising 5% FBS and 1% BSA. Staining was performed by attaching the ZO-1 primary antibody (Abcam, Cambridge, UK) for 12 hours at 4 °C, and attaching the secondary antibody (ThermoFisher Scientific, MA, USA) for 1 hour at room temperature. High-resolution fluorescence images were captured using EVOS™ FL Auto2 Imaging System (ThermoFisher Scientific, MA, USA), and quantitative analysis of fluorescence intensity was performed using Celleste Image Analysis Software (ThermoFisher Scientific, MA, USA).

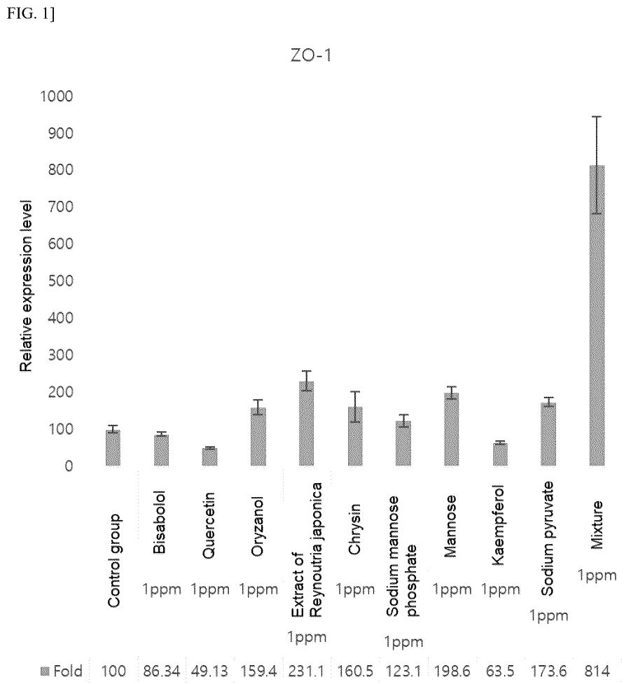

The test substances were each treated at a concentration of 1 μg/ml using bisabolol, oryzanol, extract of Reynoutria japonica, chrysin, quercetin, sodium mannose phosphate, kaempferol and pyruvate, as well as a mixture comprising these substances, as described in Table 2 below, and the measured ZO-1 expression level is shown in FIG. 1.

TABLE 2

| | Substance treated | Concentration (ppm, μg/ml) |
|---|---|---|
| 1 | Bisabolol | 1 |
| 2 | Oryzanol | 1 |
| 3 | Extract of Reynoutria japonica | 1 |
| 4 | Chrysin | 1 |
| 5 | Quercetin | 1 |
| 6 | Sodium mannose phosphate | 1 |
| 7 | Kaempferol | 1 |
| 8 | Pyruvate | 1 |
| 9 | Mixture of the above substances | 1 |

As shown in FIG. 1, each of bisabolol, oryzanol, extract of Reynoutria japonica, chrysin, quercetin, sodium mannose phosphate, kaempferol, pyruvate showed an effect on ZO-1 expression of about 0.5 to about 2.5 times that of the control group, whereas when a mixture of these was treated, an increase in ZO-1 expression of about 8 times was shown, indicating an excellent synergistic effect.

### Test Example 2-3. Evaluation of Pigmentation Suppression and Collagen Production Promotion

To evaluate the inhibitory efficacy on the expression of KITLG and MMP9, mRNA expression analysis was performed. HaCaT cells were seeded in 24-well plates and incubated at 37 °C, 5% CO₂ for 24 hours. The test substance was dissolved in 1 mL of medium and treated to the cells, and then incubated for another 24 hours at 37 °C, 5% CO₂. To extract RNA from the incubated cells, RNA prep was performed according to the manual of AccuPrep® Universal RNA Extraction Kit (BIONEER; cat #K-3140). cDNA was produced from the extracted RNA using AccuPower® RocketScript™ Cycle RT PreMix (dT20) (BIONEER; cat #K-2202). Real-time quantitative Polymerase Chain Reaction was performed with the cDNA produced in the above. Taqman universal master mix II, with UNG (ABI; cat #4440045, USA) was used, and Taqman™ probe GAPDH (reference gene), KITLG and MMP9 were used as probes. PCR reactions were performed according to the ABI7500 Real Time PCR system protocol, and data were obtained using ABI software (delta delta Ct method).

Delta Ct: Ct(target gene)−Ct(GAPDH)

Delta delta Ct: delta Ct(test substance treatment group)−delta Ct(control group)

The delta delta Ct value obtained with the above equation can be regarded as the number of gene amplifications in the test substance treatment group compared to the control group. Since the gene is amplified by the power of 2, the expression amount is calculated as 2\hat{ }(−delta delta Ct). If the expression amount in the test substance treatment group is lower than that in the control group, it is determined that the gene has the effect of suppressing its expression.

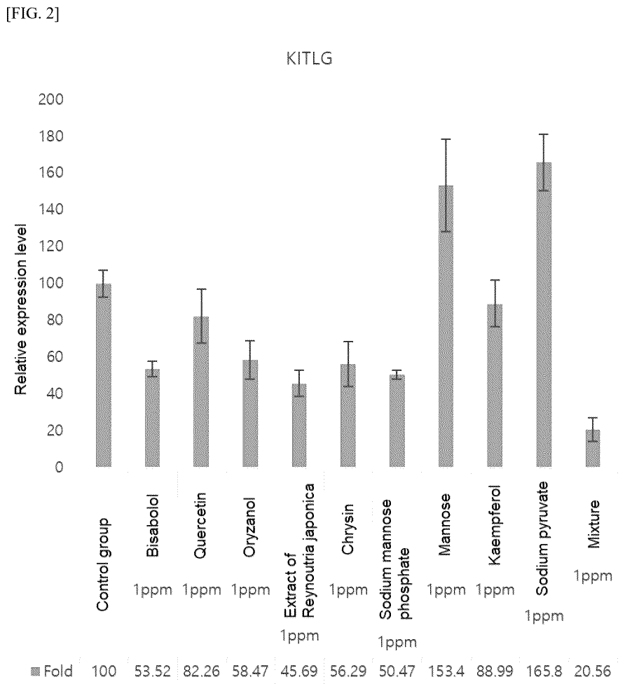

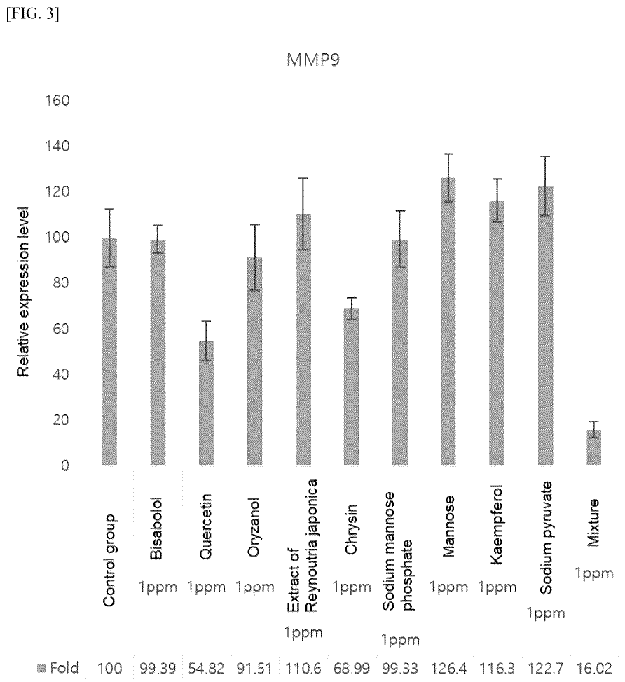

Medium without the test substance was used as the control group, and the test substances were treated with each of bisabolol, oryzanol, extract of Reynoutria japonica, chrysin, quercetin, sodium mannose phosphate, kaempferol and pyruvate, and a mixture thereof, as described in Table 2 above, to a final concentration of 1 μg/ml. The measured degree of KITLG expression inhibition is shown in FIG. 2, and the degree of MMP9 expression inhibition is shown in FIG. 3.

As shown in FIG. 2, each of bisabolol, oryzanol, extract of Reynoutria japonica, chrysin, quercetin, sodium mannose phosphate, kaempferol, and pyruvate showed KITLG expression values of about 40% to about 80% compared to the control group or even increased KITLG expression, whereas when the mixture of these was treated, the KITLG expression value showed a low value of about 20%, indicating an excellent synergistic effect.

In addition, as shown in FIG. 3, each of bisabolol, oryzanol, extract of Reynoutria japonica, chrysin, quercetin, sodium mannose phosphate, kaempferol, and pyruvate showed an MMP9 expression value of about 50% to 90% of the control group or even increased MMP9 expression, whereas when a mixture of these was treated, MMP9 expression showed a low value of less than 20%, indicating an excellent synergistic effect.

1. A cosmetic composition for improving skin conditions, comprising bisabolol, oryzanol, extract of Reynoutria japonica, chrysin, quercetin, sodium mannose phosphate, kaempferol and pyruvate.

2. The cosmetic composition of claim 1, characterized in that the weight ratio of bisabolol:oryzanol:extract of Reynoutria japonica:chrysin:quercetin:sodium mannose phosphate:kaempferol:pyruvate is 1:0.05~0.5:0.01~0.1:0.005~0.05:0.005~0.05:0.001~0.01:0.0005~0.005:0.00005~0.0005.

3. The cosmetic composition of claim 1, characterized in that the cosmetic composition comprises, as a solvent, one selected from the group consisting of water, lower alcohols with C₁ to C₄, polyhydric alcohols and mixtures thereof.

4. The cosmetic composition of claim 1, characterized in that the improving skin conditions is one or more selected from the group consisting of skin barrier strengthening, pigmentation suppression, wrinkle improvement, and elasticity enhancement.

5. A skin topical formulation for improving skin conditions, comprising bisabolol, oryzanol, extract of Reynoutria japonica, chrysin, quercetin, sodium mannose phosphate, kaempferol and pyruvate.

6. The skin topical formulation of claim 5, characterized in that the weight ratio of bisabolol:oryzanol:extract of Reynoutria japonica:chrysin:quercetin:sodium mannose phosphate:kaempferol:pyruvate is 1:0.05~0.5:0.01~0.1:0.005~0.05:0.005~0.05:0.001~0.01:0.0005~0.005:0.00005~0.0005.

7. The skin topical formulation of claim 5, characterized in that the skin topical formulation comprises, as a solvent, one selected from the group consisting of water, lower alcohols with C₁ to C₄, polyhydric alcohols and mixtures thereof.

8. The skin topical formulation of claim 5, characterized in that the improving skin conditions is one or more selected from the group consisting of skin barrier strengthening, pigmentation suppression, wrinkle improvement, and elasticity enhancement.
